(19) United States
(12) Patent Application Publication
(10) Pub. No.: US 2012/0015016 A1
(43) Pub. Date: Jan. 19, 2012

(54) UV Protecting Composition And Methods Of Use

(75) Inventors: Angelike Galdi, Westfield, NJ (US); Sidney Peter Foltis, Nutley, NJ (US); Anil Shah, East Windsor, NJ (US)

(73) Assignee: L'OREAL, Paris (FR)

(21) Appl. No.: 13/259,339

(22) PCT Filed: Apr. 12, 2010
(86) PCT No.: PCT/US10/30707

§ 371 Date: Sep. 23, 2011

Related U.S. Application Data
(60) Provisional application No. 61/168,435, filed on Apr. 10, 2009.

(51) Int. Cl. A61K 8/81; A61Q 17/04; A61K 8/02

(52) U.S. Cl. 424/401; 424/59; 424/60

(57) ABSTRACT

The present application relates to a UV protecting composition containing: (a) at least one organic UV sunscreen active; (b) at least one semi-crystalline polymer which is solid at ambient temperature and has a melting point of less than about 8O0 C; (c) hollow latex particles; and (d) at least one additional ingredient chosen from: i) a UV light absorbing compound having an SPF of less than 2, and ii) an SPF booster capable of reflecting UV light, different from hollow latex particles, wherein the hollow latex particles are employed in an amount equal to, or greater than, the amount of the at least one organic UV sunscreen active present in the composition. The invention also relates processes for protecting cellular targets from UV rays and free radical-induced damage by topically applying a UV protecting composition onto a keratinous substance, such as skin, and processes for making such compositions.

This application claims the benefit of priority under 35 U.S.C. §119(e) from U.S. provisional application Ser. No. 61/168,435, filed Apr. 10, 2009, incorporated herein by reference in its entirety.

### BACKGROUND OF THE INVENTION

Aging skin is the result of more than just chronological age. Skin is exposed to various environmental stresses, such as UV rays, which cause free radicals to form in the skin. Free radicals include, for example, singlet oxygen, hydroxyl radical, the superoxide anion, nitric oxide and hydrogen radicals. Free radicals attack DNA, membrane lipids and proteins, generating carbon radicals. These in turn react with oxygen to produce a peroxyl radical which may attack adjacent fatty acids to generate new carbon radicals. This process can lead to a chain reaction producing lipid peroxidation products. Damage to the cell membrane can result in loss of cell permeability, increased intercellular ionic concentration and/or decreased ability to excrete or detoxify waste products. The end result is a loss of elasticity of the skin and the appearance of wrinkles leading to premature ageing of the skin. This process is commonly referred to as photo-aging.

Generally, UV sunscreen actives are utilized to provide protection from UV light. Greater levels of UV protection typically require larger amount of actives to be employed. Some consumers, however, experience skin sensitivity issues when exposed to organic sunscreen actives.

Thus, it is an object of the present invention to provide a composition and process for providing maximum protection of cellular targets from aging and photo-damage caused by UV light, in general, and free radicals formed thereby, in particular, while simultaneously employing minimal amounts of organic sunscreen actives, thereby decreasing skin sensitivity issues.

### SUMMARY OF THE INVENTION

The present invention is directed to a UV protecting composition containing: (a) at least one organic UV sunscreen active; (b) at least one semi-crystalline polymer which is solid at ambient temperature and has a melting point of less than about 80° C.; (c) hollow latex particles; and (d) at least one additional ingredient chosen from: i) a UV light absorbing compound having an SPF of less than 2, and ii) an SPF booster capable of reflecting UV light, different from hollow latex particles, wherein the hollow latex particles are employed in an amount equal to, or greater than, the amount of the at least one organic UV sunscreen active present in the composition.

The present invention is also directed to a process for protecting cellular targets from UV rays and free radical-induced damage by topically applying the above-disclosed composition onto keratinous tissue, such as skin. The process for protecting includes both prophylactic and therapeutic inhibition of free radical-induced damage of the cellular targets.

Surprisingly, the applicants have discovered that, by combining an organic UV screening agent, a semi-crystalline polymer, hollow latex particles, and an additional UV-active ingredient, different from the hollow latex particles, such that the amount of hollow latex particles is equal to or greater than the amount of organic UV screening agent, it is possible to produce a composition with broad spectrum UVB/UVA protection having a desirable Sun Protection Factor (“SPF”), e.g., from about 15 to about 70. Unexpectedly, the compositions of the invention achieve these effects while employing much less of the organic UV screening agent than do conventional sunscreen products.

### BRIEF DESCRIPTION OF THE FIGURES

FIG. 1 is a bar graph evidencing the p53 efficacy of the present invention.

FIG. 2 is a bar graph evidencing the sunburn cell efficacy of the present invention.

### DETAILED DESCRIPTION

Other than in the operating examples, and where otherwise indicated, all numbers expressing quantities of ingredients and/or reaction conditions are to be understood as being modified in all instances by the term “about,” meaning within 10% to 15% of the indicated number.

In the present application the term “ambient temperature” means a temperature of 25° C.

A “physiologically acceptable medium” means a medium which is not toxic and can be applied to the skin, lips, hair, scalp, lashes, brows, nails or any other cutaneous region of the body. The composition of the invention may especially constitute a cosmetic or dermatological composition.

### Organic UV Sunscreen Actives

The UV protecting composition of the invention contains organic UV sunscreen actives.

The organic UV sunscreen actives are selected from water-soluble organic sunscreen actives, fat-soluble organic sunscreen actives or agents which are insoluble in the solvents presently used in suntan products, and mixtures thereof.

The organic UV sunscreen actives are especially selected from cinnamic derivatives; anthranilates; salicylic derivatives; dibenzoylmethane derivatives; camphor derivatives; benzophenone derivatives; β,β-diphenylacrylate derivatives; triazine derivatives; benzotriazole derivatives; benzalmalonate derivatives, especially those cited in patent U.S. Pat. No. 5,624,663; benzimidazole derivatives; imidazolines; bis-benzazolyl derivatives such as those described in patents EP669323 and U.S. Pat. No. 2,463,264; p-aminobenzoic acid (PABA) derivatives; methylenebis(hydroxyphenylbenzotriazole) derivatives such as those described in patent applications U.S. Pat. No. 5,237,071, U.S. Pat. No. 5,166,355, GB2303549, DE 197 26 184 and EP893119; benzoxazole derivatives such as those described in patent applications EP0832642, EP1027883, E21300137 and DE10162844; screening polymers and screening silicones such as those described especially in patent application WO 93/04665; α-alkylstyrene-derived dimers such as those described in patent application DE19855649; 4,4-diarylbutadienes such as those described in patent applications EP0967200, DE19746654, DE19755649, EP-A-1008586, EP1133980 and EP133981; merocyanin derivatives such as those described in patent applications WO 04/006878, WO 05/058269 and WO 06/032741; and mixtures thereof.

Examples of complementary organic photoprotective agents include those denoted hereinbelow under their INCI name:

### Cinnamic Derivatives:

Ethylhexyl Methoxycinnamate sold in particular under the trade name “PARSOL MOX” by Hoffmann LaRoche,

### Isopropyl Methoxycinnamate,

Isoamyl Methoxycinnamate sold under the trade name “NEO HELIOPAN E 1000” by Haarmann and Reimer,

### DEA Methoxycinnamate,

### Diisopropyl Methylcinnamate,

### Glyceryl Ethylhexanoate Dimethoxycinnamate.

### Dibenzoylmethane Derivatives:

Butyl Methoxydibenzoylmethane (also known as avobenzone) sold especially under the trade name “PARSOL 1789” by Hoffmann LaRoche,

### Isopropyl Dibenzoylmethane.

### Para-Aminobenzoic Acid Derivatives:

### PABA,

### Ethyl PABA,

### Ethyl Dihydroxypropyl PABA,

Ethylhexyl Dimethyl PABA sold in particular under the name “ESCALOL 507” by ISP,

### Glyceryl PABA,

PEG-25 PABA sold under the name “UVINUL P25” by BASF.

### Salicylic Derivatives:

Homosalate sold under the name “EUSOLEX HMS” by Rona/EM Industries,
Ethylhexyl Salicylate sold under the name “NFO HELIOPAN OS” by Haarmann and Reimer,
Dipropylene Glycol Salicylate sold under the name “DIPSAL” by Scher,
TEA Salicylate sold under the name “NEO HELIOPAN TS” by Haarmann and Reimer.

### β,β-Diphenylacrylate Derivatives:

Octocrylene sold in particular under the trade name “UVINUL N539” by BASF,
Etocrylene sold in particular under the trade name “UVINUL N35” by BASF.

### Benzophenone Derivatives:

Benzophenone-1 sold under the trade name “UVINUL 400” by BASF,
Benzophenone-2 sold under the trade name “UVINUL D50” by BASF,
Benzophenone-3 or Oxybenzone sold under the trade name “UVINUL M40” by BASF,
Benzophenone-4 sold under the trade name “UVINUL MS40” by BASF,

### Benzophenone-5,

Benzophenone-6 sold under the trade name “HELISORB 11” by Norquay,
Benzophenone-8 sold under the trade name “SPECTRA-SORB UV-24” by American Cyanamid,
Benzophenone-9 sold under the trade name “UVINUL DS-49” by BASF,

### Benzophenone-12,

n-Hexyl 2-(4-diethylamino-2-hydroxybenzoyl)benzoate sold under the trade name “UVINUL A+” by BASF.

### Benzylidenecamphor Derivatives:

3-Benzylidenecamphor manufactured under the name “MEXORYL SD” by Chimex,
4-Methylbenzylidenecamphor sold under the name “EUSOLEX 6300” by Merck,
Benzylidene Camphor Sulfonic acid manufactured under the name “MEXORYL SL” by Chimex,
Camphor Benzalkonium Methosulfate manufactured under the name “MEXORYL SO” by Chimex,
Terephthalylidene Dicamphor Sulfonic acid manufactured under the name “MEXORYL SX” by Chimex,
Polyacrylamidomethyl Benzylidene Camphor manufactured under the name “MEXORYL SW” by Chimex.

### Phenylbenzimidazole Derivatives:

Phenylbenzimidazole Sulfonic acid sold in particular under the trade name “EUSOLEX 232” by Merck, Disodium Phenyl Dibenzimidazole Tetrasulfonate sold under the trade name “NEO HELIOPAN AP” by Haarmann and Reimer.

### Phenylbenzotriazole Derivatives:

Drometrizole Trisiloxane sold under the name “SILATRIZOLE” by Rhodia Chimie,
Methylene bis(Benzotriazolyl) Tetramethylbutylphenol sold in solid form under the trade name “MIXXIM BB/100” by Fairmount Chemical, or in micronized form as an aqueous dispersion under the trade name “TINOSORB M” by Ciba Specialty Chemicals.

### Triazine Derivatives:

bis-Ethylhexyloxyphenol Methoxyphenyl Triazine sold under the trade name “TINOSORB S” by Ciba Geigy,

Ethylhexyl Triazone sold in particular under the trade name “UVINUL T150” by BASF,

Diethylhexyl Butamido Triazone sold under the trade name “UVASORB HEB” by Sigma 3V,

2,4,6-Tris(dineopentyl 4′-aminobenzalmalonate)-s-triazine,

2,4,6-Tris(diisobutyl 4′-aminobenzalmalonate)-s-triazine,

2,4-Bis(n-butyl 4′-aminobenzoate)-6-(aminopropyltri-siloxane)-s-triazine,

2,4-Bis(dineopentyl 4′-aminobenzalmalonate)-6-(n-butyl 4′-aminobenzoate)-s-triazine,

the symmetrical triazine sunscreen actives described in patent U.S. Pat. No. 6,225,467, patent application WO 2004/085412 (see compounds 6 and 9) or the document “Symmetrical Triazine Derivatives” IP.COM Journal, IP.COM Inc., West Henrietta, N.Y., US (20 Sep. 2004), especially 2,4,6-tris(biphenyl-1,3,5-triazines (in particular 2,4,6-tris(biphenyl-4-yl)-1,3,5-triazine and 2,4,6-tris(terphenyl)-1,3,5-triazine, which is found in the patent applications WO 06/035000, WO 06/034982, WO 06/034991, WO 06/035007, WO 2006/034992 and WO 2006/034985.

### Anthranilic Derivatives:

Menthyl anthranilate sold under the trade name “NEO HELIOPAN MA” by Haarmann and Reimer.

### Imidazoline Derivatives:

### Ethylhexyl Dimethoxybenzylidene Dioxoimidazoline Propionate.

### Benzalmalonate Derivatives:

Polyorganosiloxane containing benzalmalonate functions, for instance Polysilicone-15, sold under the trade name “PARSOL SLX” by Hoffmann LaRoche.

### 4,4-Diarylbutadiene Derivatives:

1,1-Dicarboxy(2,2′-dimethylpropyl)-4,4-diphenylbutadiene.

### Benzoxazole Derivatives:

2,4-Bis[5-(1-dimethylpropyl)benzoxazol-2-yl(4-phenyl)imino]-6-(2-ethylhexyl)imino-1,3,5-triazine sold under the name UVASORB K2A by Sigma 3V,
and mixtures thereof.
The preferential organic sunscreen actives are selected from:
Ethylhexyl Methoxycinnamate,
Ethylhexyl Salicylate,
Homosalate,
Butyl Methoxydibenzoylmethane,
Octocrylene,
Phenylbenzimidazole Sulfonic Acid,
Benzophenone-3,
Benzophenone-4,
Benzophenone-5,
n-Hexyl 2-(4-diethylamino-2-hydroxybenzoyl)benzoate,
4-Methylbenzylidene camphor,
Terephthalylidene Dicamphor Sulfonic Acid,
Disodium Phenyl Dibenzimidazole Tetrasulfonate,
Methylene bis-Benzotriazolyl Tetramethylbutylphenol,
Bis-Ethylhexyloxyphenol Methoxyphenyl Triazine,
Ethylhexyl Triazone,
Diethylhexyl Butamido Triazone,
2,4,6-Tris(dineopentyl 4′-aminobenzalmalonate)-s-triazine,
2,4,6-Tris(diisobutyl 4′-aminobenzalmalonate)-s-triazine,
2,4-Bis(n-butyl 4′-aminobenzoate)-6-(aminopropyltrisiloxane)-s-triazine,
2,4-Bis(dineopentyl 4ζ-aminobenzalmalonate)-6-(n-butyl 4′-aminobenzoate)-s-triazine,
2,4,6-Tris(biphenyl-4-yl)-1,3,5-triazine,
2,4,6-Tris(terphenyl)-1,3,5-triazine,
Drometrizole Trisiloxane,
Polysilicone-15,
1,1-Dicarboxy(2,2′-dimethylpropyl)-4,4-diphenylbutadiene,
2,4-Bis[5-1-(dimethylpropyl)benzoxazol-2-yl(4-phenyl)imino]-6-(2-ethylhexyl)-imino-1,3,5-triazine,
   and mixtures thereof.

Organic UV sunscreen actives are typically employed in an amount of from about 1 to about 15% by weight, such as from about 1 to about 12% by weight, from about 1 to about 10% by weight, from about 1 to about 8% by weight, from about 1 to about 6% by weight, and from about 1 to about 4% by weight, based on the total weight of the composition, including all ranges and subranges therebetween.

### Semi-Crystalline Polymers

The semi-crystalline polymer used in the composition of the invention allows higher protection indices to be obtained without an increase in the level of chemical sunscreen actives, and hence makes it possible to improve the protection index for a given quantity of screening agent. The polymer is generally introduced into the liquid fatty phase (also referred to below as oily phase).

“Polymers” for the purposes of the invention are compounds containing at least 2 repeating units, preferably at least 3 repeating units and more especially at least 10 repeating units.

“Semi-crystalline polymer” for the purposes of the invention means polymers comprising a crystallizable part, a crystallizable pendant chain or crystallizable block in the backbone, and an amorphous part in the backbone, and exhibiting a first-order reversible phase-change temperature, more particularly melting (solid-liquid transition) temperature. When the crystallizable part is in the form of a crystallizable block of the polymeric backbone, the amorphous part of the polymer is in the form of an amorphous block; in that case the semi-crystalline polymer is a block copolymer, of the diblock, triblock or multiblock type, for example, comprising at least one crystallizable block and at least one amorphous block.

By “block” is meant, generally, at least 5 identical repeating units. In that case the crystallizable block or blocks are different in chemical nature from the amorphous block or blocks.

A “crystallizable chain or block” for the purposes of the invention is a chain or block which, on its own, would pass from the amorphous state to the crystalline state reversibly according to whether it were above or below the melting temperature. A chain for the purposes of the invention is a group of atoms which is pendant or lateral relative to the backbone of the polymer. A block is a group of atoms that belongs to the backbone, a group constituting one of the repeating units of the polymer. Advantageously the “crystallizable pendant chain” may be a chain containing at least 6 carbon atoms.

The semi-crystalline polymer according to the invention has a melting temperature of greater than or equal to 30° C., preferably ranging from 30° C. to 80° C., and more preferably ranging from 30° C. to 70° C. This melting temperature is a first-order state-change temperature. This melting temperature may be measured by any known method and more particularly by using a differential scanning calorimeter (DSC).

Advantageously the semi-crystalline polymer or polymers to which the invention applies exhibit a number-average molecular mass of greater than or equal to 1000.

Advantageously the semi-crystalline polymer or polymers of the composition of the invention have a number-average molecular mass M̅n ranging from 2,000 to 800,000, preferably from 3,000 to 500,000, better still from 4,000 to 150,000, especially less than 100,000, and better still from 4,000 to 99,000. Preferably they exhibit a number-average molecular mass of greater than 5,600, ranging for example from 5,700 to 99,000.

Preferably the crystallizable block or blocks or chain or chains of the semi-crystalline polymers represent at least 30% of the total weight of each polymer and better still at least 40%. The semi-crystalline polymers of the invention having crystallizable blocks are block or multiblock polymers. They may be obtained by polymerization of monomer having reactive (or ethylenic) double bonds or by polycondensation. When the polymers of the invention are polymers having crystallizable side chains, these polymers are advantageously in random or statistical form.

The semi-crystalline polymers of the invention are preferably synthetic in origin. Moreover, they do not contain a polysaccharide backbone. Generally speaking, the crystallizable units (chains or blocks) of the semi-crystalline polymers according to the invention originate from one or more monomers having crystallizable blocks or chains, which are used for the preparation of the semi-crystalline polymers.

According to the invention the semi-crystalline polymers are selected from block copolymers containing at least one crystallizable block and at least one amorphous block, the homopolymers and the copolymers bearing at least one crystallizable side chain per repeating unit, and mixtures thereof.

The semi-crystalline polymers which can be used in the invention are more particularly:

block polyolefin copolymers having controlled crystallization, especially those whose monomers are described in EP-A-0 951 897;

polycondensates, and especially those of aliphatic or aromatic polyester or aliphatic/aromatic copolyester type;

homopolymers or copolymers bearing at least one crystallizable side chain, and homopolymers or copolymers bearing in the backbone at least one crystallizable block, such as those described in document U.S. Pat. No. 5,156,911;

homopolymers or copolymers bearing at least one crystallizable side chain, more particularly having one or more fluorinated groups, such as those described in document WO-A-01/19333, and mixtures thereof. In these last two cases the crystallizable block or blocks or side chain or side chains are hydrophobic.

### A) Semi-Crystalline Polymers Having Crystallizable Side Chains

Mention may be made more particularly of those defined in document U.S. Pat. No. 5,156,911 and WO-A-01/19333. These are homopolymers or copolymers containing from 50% to 100% by weight of units resulting from the polymerization of one or more monomers bearing a crystallizable hydrophobic side chain.

These homopolymers or copolymers are of any kind, provided that they meet the conditions set out above.

They may result:

from the polymerization, especially free-radical polymerization, of one or more monomers having one or more double bonds which are reactive or ethylenic in relation to a polymerization, namely a vinyl, (meth)acrylic or allyl group;

from the polycondensation of one or more monomers which bear co-reactive groups (carboxylic or sulfonic acid, alcohol, amine or isocyanate), such as, for example, polyesters, polyurethanes, polyethers, polyureas and polyamides.

Generally speaking, these polymers are selected especially from homopolymers and copolymers resulting from the polymerization of at least one monomer which has one or more crystallizable chains and may be represented by the formula X:

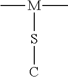

with M representing an atom of the polymeric backbone, S representing a spacer and C representing a crystallizable group.

The crystallizable chains “S—C” may be aliphatic or aromatic and optionally fluorinated or perfluorinated. “S” represents especially a linear or branched or cyclic (CH₂)ₙ or (CH₂CH₂O)ₙ or (CH₂O) group, with n being an integer ranging from 0 to 22. Preferably “S” is a linear group. Preferably “S” and “C” are different.

When the crystallizable chains “S—C” are aliphatic hydrocarbon chains, they comprise hydrocarbon alkyl chains having at least 11 carbon atoms and not more than 40 carbon atoms and better still not more than 24 carbon atoms. They are, especially, aliphatic chains or alkyl chains possessing at least 12 carbon atoms, and preferably they are C₁₄-C₂₄ alkyl chains. When they are fluorinated or perfluorinated alkyl chains, they contain at least 6 fluorinated carbon atoms and especially at least 11 carbon atoms of which at least 6 carbon atoms are fluorinated.

Examples of semi-crystalline polymers or copolymers having one or more crystallizable chains include those resulting from the polymerization of one or more of the following monomers: saturated alkyl (meth)acrylates with a C₁₄-C₂₄ alkyl group, perfluoroalkyl (meth)acrylates with a C₁₁-C₁₅ perfluoroalkyl group, N-alkyl(meth)acrylamides with a C₁₄ to C₂₄ alkyl group with or without a fluorine atom, vinyl esters having alkyl or perfluoro(alkyl) chains with a C₁₄ to C₂₄ alkyl group (with at least 6 fluorine atoms for a perfluoroalkyl chain), vinyl ethers having alkyl or perfluoro(alkyl) chains with a C₁₄ to C₂₄ alkyl group and at least 6 fluorine atoms for a perfluoroalkyl chain, C₁₄ to C₂₄ alpha-olefins such as, for example, octadecene, para-alkylstyrenes with an alkyl group containing 12 to 24 carbon atoms, and mixtures thereof.

When the polymers result from a polycondensation, the crystallizable hydrocarbon and/or fluorinated chains as defined above are borne by a monomer which may be a diacid, a diol, a diamine or a diisocyanate.

When the polymers that are subject matter of the invention are copolymers, they contain, in addition, from 0% to 50% of groups Y or Z resulting from the copolymerization: of Y, which is a polar or non-polar monomer or a mixture of the two:

When Y is a polar monomer, it is alternatively a monomer which carries polyalkoxylated groups (especially ethoxylated and/or propoxylated groups), a hydroxyalkyl (meth)acrylate such as hydroxyethyl acrylate, (meth)acrylamide, an N-alkyl(meth)acryiamide, an N,N-dialkyl(meth)acrylamide such as, for example, N,N-diisopropylacrylamide, or N-vinylpyrrolidone

(NVP), N-vinylcaprolactam, a monomer which carries at least one carboxylic acid group, such as (meth)acrylic, crotonic, itaconic, maleic or fumaric acids, or which carries a carboxylic anhydride group, such as maleic anhydride, and mixtures thereof.

When Y is a non-polar monomer it may be an ester of the linear, branched or cyclic alkyl (meth)acrylate type, a vinyl ester, an alkyl vinyl ether, an alpha-olefin, styrene or a styrene substituted by a C₁ to C₁₀ alkyl group, such as a-methylstyrene, or a macromonomer of the polyorganosiloxane type having vinylic unsaturation.

“Alkyl” for the purposes of the invention means a saturated group which especially is a C₈ to C₂₄ group, unless expressly mentioned, and better still a C₁₄ to C₂₄ group.

b) of Z, which is a polar monomer or a mixture of polar monomers.

In this case, Z has the same definition as the “polar Y” defined above.

Preferably the semi-crystalline polymers having a crystallizable side chain are homopolymers of alkyl (meth)acrylate or alkyl(meth)acrylamide with an alkyl group as defined above, and more particularly a C₁₄-C₂₄ group, and copolymers of these monomers with a hydrophilic monomer which is preferably different in nature from (meth)acrylic acid, such as N-vinylpyrrolidone or hydroxyethyl (meth)acrylate and mixtures thereof.

### B) Polymers Bearing in the Backbone at Least One Crystallizable Block

These polymers are especially block copolymers constituted of at least two chemically different blocks of which one is crystallizable.

Use may be made of the block polymers defined in patent U.S. Pat. No. 5,156,911;

olefin or cycloolefin block copolymers having crystallizable chains, such as those obtained from the block polymerization of:

cyclobutene, cyclohexene, cyclooctene, norbornene (i.e. bicyclo[2.2.1]hept-2-ene), 5-methylnorbornene, 5-ethyl-norbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8,8a-octahydronaphthalene, dicyclopentadiene or mixtures thereof, with ethylene, propylene, 1-butene, 3-methyl-1-butene, 1-hexene, 4-methyl-1-pentene, 1-octene, 1-decene, 1-eicosene or mixtures thereof, and more particularly block copoly(ethylene/norbornene)s and block (ethylene/propylene/ethylidene-norbornene) terpolymers. Use may also be made of those resulting from the block copolymerization of at least two C₂-C₁ and better still C₂-C₁₂ and even better still C₄-C₁₂ α-olefins, such as those referred to above, and more particularly the block bipolymers of ethylene and 1-octene.

The copolymers may be copolymers exhibiting at least one crystallizable block, the remainder of the copolymer being amorphous (at ambient temperature). These copolymers may, moreover, exhibit two crystallizable blocks differing in chemical nature. The preferred copolymers are those which at ambient temperature possess both a crystallizable block and an amorphous block, both hydrophobic and lipophilic, which are distributed sequentially; mention may be made, for example, of polymers possessing one of the following crystallizable blocks and one of the following amorphous blocks:

Naturally crystallizable block: a) polyester such as poly(alkylene terephthalate)s, b) polyolefin such as polyethylenes or polypropylenes.

Amorphous and lipophilic block, for instance amorphous polyolefins or copoly(olefin)s, such as poly(isobutylene), hydrogenated polybutadiene and hydrogenated poly(isoprene).

Examples of such copolymers having a separated crystallizable block and amorphous block include the following:

a) block poly(c-caprolactone)-b-poly(butadiene) copolymers, preferably used in hydrogenated form, such as those described in the article “Melting behavior of poly(ε-caprolactone)-block-polybutadiene copolymers” by S, Nojima, Macromolecules, 32, 3727-34 (1999).
b) block or multiblock hydrogenated poly(butylene terephthalate)-b-poly(isoprene) block copolymers, as cited in the article “Study of morphological and mechanical properties of PP/PBT” by P. Boutevin et al., Polymer Bulletin, 34, 117-23 (1995).
c) the poly(ethylene)-b-copoly(ethylene/propylene) block copolymers that are cited in the articles “Morphology of semi-crystalline block copolymers of ethylene-(ethylene-alt-propylene)” by P. Rangarajan et al., Macromolecules, 26, 4640-45 (1993) and “Polymer aggregates with crystalline cores: the system poly(ethylene)-poly(ethylene-propylene)” by P. Richter et al., Macromolecules, 30, 1053-68 (1997).
d) the poly(ethylene)-b-poly(ethylethylene) block copolymers that are cited in the general article “Crystallization in block copolymers” by I. W. Hamley, Advances in Polymer Science, vol. 148, 113-37 (1999).

The semicrystalline polymers of the composition of the invention may be non-crosslinked or partly crosslinked, with the proviso that the degree of crosslinking is not disruptive to their dissolution or dispersion in the liquid fatty phase by heating to above their melting temperature. In that case the crosslinking in question may be chemical crosslinking, by reaction with a polyfunctional monomer during the polymerization. It may also be physical crosslinking, which in that case may be due either to the establishment of hydrogen bonds or dipole bonds between groups borne by the polymer, such as, for example, dipolar interactions between carboxylate ionomers, these interactions being small in quantity and borne by the backbone of the polymer, or to phase separation between the crystallizable blocks and the amorphous blocks which are borne by the polymer.

Preferably the semi-crystalline polymers of the composition according to the invention are non-crosslinked.

According to one particular embodiment of the invention the polymer is selected from copolymers resulting from the polymerization of at least one monomer having a crystallizable chain and selected from saturated C₁₄ to C₂₄ alkyl (meth)acrylates, C₁₁ to C₁₅ perfluoroalkyl (meth)acrylates, C₁₄ to C₂₄ N-alkyl(meth)acrylamides with or without a fluorine atom, vinyl esters having C₁₄ to C₂₄ alkyl or perfluoroalkyl chains, vinyl ethers having C₁₄ to C₂₄ alkyl or perfluoroalkyl chains, C₁₄ to C₂₄ alpha-olefins, para-alkylstyrenes with an alkyl group containing 12 to 24 carbon atoms, with at least one ester or amide of C₁ to C₁₀ monocarboxylic acid which is optionally fluorinated, which may be represented by the formula below:

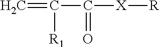

in which R₁ is H or CH₃, R represents an optionally fluorinated C₁-C₁₀ alkyl group and X represents O, NH or NR₂, where R₂ represents an optionally fluorinated C₁-C₁₀ alkyl group.

Advantageously the semi-crystalline polymer present in the composition according to the invention is not a polycaprolactone.

As a particular example of a structuring semi-crystalline polymer which can be used in the composition according to the invention, mention may be made of the Intelimer® products from the company Landec which are described in the brochure “Intelimer® polymers”, Landec IP22 (version 4-97). These polymers are in solid form at ambient temperature (25° C.) They bear crystallizable side chains and have the above formula X.

The semi-crystalline polymers may especially be: those described in Examples 3, 4, 5, 7, 9 and 13 of patent U.S. Pat. No. 5,156,911, having a —COOH group resulting from the copolymerization of acrylic acid and C₅ to C₁₆ alkyl (meth)acrylate and more particularly from the copolymerization:

of acrylic acid, hexadecyl acrylate and isodecyl acrylate in a 1/16/3 weight ratio;

of acrylic acid and pentadecyl acrylate in a 1/19 weight ratio,

of acrylic acid, hexadecyl acrylate and ethyl acrylate in a 2.5/76.5/20 weight ratio,

of acrylic acid, hexadecyl acrylate and methyl acrylate in a 5/85/10 weight ratio,

of acrylic acid and octadecyl methacrylate in a 2.5/97.5 weight ratio,

of hexadecyl acrylate, polyethylene glycol methacrylate monomethyl ether containing 8 ethylene glycol units, and acrylic acid, in an 8.5/1/0.5 weight ratio.

It is also possible to use “Structure 0” from National Starch, such as that described in document U.S. Pat. No. 5,736,125, with a melting point of 44° C., and also semi-crystalline polymers having crystallizable pendant chains containing fluorinated groups, such as those described in Examples 1, 4, 6, 7 and 8 of document WO-A-01/19333.

It is additionally possible to use low-melting-point semi-crystalline polymers obtained by copolymerizing stearyl acrylate and acrylic acid or NVP, as described in document U.S. Pat. No. 5,519,063 or EP-A-550745, and more especially those described in Examples 1 and 2, below, which are polymer preparation examples, with a melting temperature, respectively, of 40° C. and 38° C.

It is also possible to use semi-crystalline polymers obtained by copolymerizing behenyl acrylate and acrylic acid or NVP, such as those described in documents U.S. Pat. No. 5,519,063 and EP-A-550745.

Preferably the semi-crystalline polymers of low melting point and/or of high melting point do not contain a carboxyl group.

According to one more particular embodiments of the invention the polymer is the product of a monomer having a crystallizable chain, selected from saturated C₁₄ to C₂₂ alkyl (meth)acrylates, and even more particularly poly(stearyl acrylate)s or poly(behenyl acrylate)s. More particularly a selection will be made of the product Intelimer® IPA 13-1 from the company Landec, which is a polystearyl acrylate whose molecular weight is approximately 145,000 and whose melting temperature is 49° C.

Semi-crystalline polymers are typically employed in an amount of from about 0.1 to about 4% by weight, such as from about 0.2 to about 3% by weight, from about 0.2 to about 2% by weight, and from about 0.2 to about 1% by weight, based on the total weight of the composition, including all ranges and subranges therebetween.

### Hollow Latex Particles

Hollow latex particles according to the invention may be obtained from particles comprising at least one polymer for the core and at least one polymer for the shell. The core polymer and the shell polymer may be obtained from a single polymerization step or from a sequence of polymerization steps.

The hollow latex particles according to the invention have a particle size which ranges generally from 100 to 380 nm and preferably from 150 to 375 nm and more preferably from 190 to 350 nm and more particularly from 251 to 325 nm, the particle size being measured by a Brookhaven BI-90 photon correlation spectrometer.

For a given particle size, the latex particles according to the invention must in general possess a maximum hallow fraction. The latex particles preferably contain a void fraction of 0.1% to 50% and more preferably of 5% to 50%. The void fractions are determined by comparing the volume occupied by the latex particles after having been compacted from a diluted dispersion in a centrifuge, relative to the volume of non-void particles in the same composition.

Typically, the hollow latex particles are provided as part of an aqueous dispersion that is generally stabilized with at least one emulsifier.

The hollow latex particles according to the invention may be prepared by the conventional techniques of emulsion polymerization. Such processes are described especially in patents U.S. Pat. No. 4,427,836, U.S. Pat. No. 4,469,825, U.S. Pat. No. 4,594,363, U.S. Pat. No. 4,677,003, U.S. Pat. No. 4,920,160 and U.S. Pat. No. 4,970,241 or by the conventional techniques of polymerization that are described in the following patents and patent applications: EP267726, EP331421, U.S. Pat. No. 490,229 and U.S. Pat. No. 5,157,084.

The monomers used for the shell of the latex particles are preferably constituted of one or more unsaturated nonionic ethylenic units. Optionally one or more monoethylenically unsaturated monomers containing at least one carboxylic acid group may be polymerized in the shell.

The monomers constituting the shell are selected such that they exhibit a glass transition temperature (Tg) which is sufficiently high to withstand the void of the hollow latex particle. Preferably the glass transition temperature is greater than 50° C., more preferably greater than 60° C. and more preferably still greater than 70° C. This temperature Tg may be determined by DSC (differential scanning calorimetry).

The monomers used in the emulsion polymerization in the core polymer of the latex particles of the invention are preferably constituted of one or more monoethylenically unsaturated monomers containing at least one carboxylic acid group. Preferably the core comprises at least 5% by weight of monoethylenically unsaturated monomer containing at least one carboxylic acid group, relative to the total weight of the core monomers.

The core polymer may for example be obtained by emulsion homopolymerization of the monoethylenically unsaturated monomer containing at least one acid group or by copolymerization of two or three monoethylenically unsaturated monomers containing at least one acid group. Preferably the monoethylenically unsaturated monomer containing at least one acid group is copolymerized with one or more ethylenically unsaturated nonionic monomers.

The core polymer or the shell polymer may contain from 0.1% to 20% by weight, preferably from 0.1% to 3% by weight, of polyethylenically unsaturated monomers such as ethylene glycol di(meth)acrylate, allyl (meth)acrylate, 1,3-butanediol di(meth)acrylate, diethylene glycol di(meth)acrylate, trimethylolpropane tri(meth)acrylate or divinylbenzene, relative to the total weight of core monomers. Alternatively the core polymer or the shell polymer may optionally contain from 0.1% to 60% by weight of butadiene, relative to the total weight of core monomers.

The monoethylenically unsaturated monomers containing at least one carboxylic acid group include, for example: acrylic acid, methacrylic acid, acryloyloxypropionic acid, (meth)acryloyloxypropionic acid, itaconic acid, aconitic acid, maleic acid or maleic anhydride, fumaric acid, crotonic acid, monomethyl maleate, monomethyl fumarate and monomethyl itaconate.

In an embodiment, the monomer is selected from acrylic acid and methacrylic acid.

The monoethylenically unsaturated nonionic monomers include, for example: styrene, vinyltoluene, vinyl acetate, vinyl chloride, vinylidene chloride, acrylonitrile, (meth)acrylamide, C₁-C₂₀ alkyl esters of (meth)acrylic acid and (C₃-C₂₀) alkenyl esters of (meth)acrylic acid, such as methyl (meth)acrylate, ethyl (meth)acrylate, butyl (meth)acrylate, 2-ethylhexyl (meth)acrylate, benzyl (meth)acrylate, lauryl (meth)acrylate, oleyl (meth)acrylate, palmityl (meth)acrylate and stearyl (meth)acrylate. According to the invention, the term (meth)acrylic will denote the general expression encompassing both methacrylic or acrylic. The term (meth)acrylate will denote the general expression encompassing both methacrylate or acrylate.

The void part of the core of the latex particles is preferably produced by swelling the core with a swelling agent comprising one or more volatile compounds. The agent penetrates the shell in order to swell the core. The volatile components of the swelling agent may be subsequently removed by drying the latex particles, thus creating a void within the particles. The agent is preferably an aqueous base. Mention may be made, for example, of ammonia, ammonium hydroxide, alkali metal hydroxides such as sodium hydroxide and volatile amines such as trimethylamine or triethylamine.

The hollow latex particles may be introduced into the composition of the invention with the swelling agent. In that case the volatile compounds are removed when the composition is dried. The hollow latex particles may also be added to the composition after the volatile compounds of the swelling agent have been removed.

The hollow latex particles which can be used according to the invention are those described in patent U.S. Pat. No. 5,663,213 and patent application EP1092421.

According to one particular embodiment of the invention the hollow latex particles used will be those constituted of a copolymer of styrene and (meth)acrylic acid or one of its C₁-C₂₀ alkyl esters under the INCI name Styrene/Acrylates Copolymer, such as the product sold under the trade name Sunspheres™ Powder by the company Rohm & Haas, which is an aqueous dispersion containing 86% of Styrene/Acrylates Copolymer in a mixture of 11% of PEG-8 Laurate, 2.5% of water and 0.5% of Sodium Dodecylbenzenesulfonate.

The hollow latex polymers are typically employed in an amount of from about 1 to about 20% by weight, such as from about 3 to about 17% by weight, from about 5 to about 15% by weight, and from about 7 to about 10% by weight, based on the total weight of the composition, including all ranges and subranges therebetween.

In the compositions of the present invention, the hollow latex particles are employed in an amount equal to, or greater than, the amount of the at least one organic UV sunscreen active present in the composition. Applicants have discovered that when the hollow latex particles are employed in an amount equal to, or greater than, the amount of the at least one organic UV sunscreen active in the compositions of the present invention it is possible to achieve broad spectrum UVB/UVA protection and a very desirable SPF, e.g., in the range of from about 15 to about 70, while employing a minimal amount of the organic UV screening agent.

Typically, the ratio of organic UV screening agent to hollow latex polymers is in the range of from about 0.5:about 1 to about 1:about 20, such as from about 0.1:about 1 to about 1:about 15.

### Additional UV Light Absorbing Compound or SPF Booster

The compositions of the present invention contain at least one additional ingredient chosen from: i) a UV light absorbing compound having an SPF of less than 2, and ii) an SPF booster capable of reflecting UV light, different from the hollow latex particles.

The term “UV light absorbing compound having an SPF of less than 2” means a compound, other than a UV screening agent, which absorbs UV light in the range of from about 280 nm to about 400 nm (i.e., UVB/UVA). Examples of compounds capable of absorbing UV light having an SPF of less than 2 include butyloctyl salicylate, diethylhexyl syringylidenemalonate (INCI name) having the following formula:

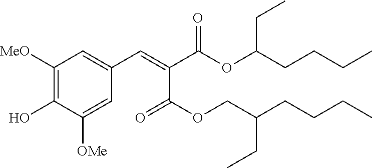

(commercially available from Merck KgaA under the trade name Oxynex® ST), diethylhesyl 2,6-naphthalate (commercially available from HallStar Co. under the trade name Corapan TQ® ST), octadecyl di-t-butyl-4-hydroxyhydrocinnamate (commercially available from Ciba Specialty Chemicals (part of BASF Corp.) under the trade name Tinogard® TS), sodium benzotriazolyl butylphenol sulfonate (commercially available from Ciba Specialty Chemicals (part of BASF Corp.) under the trade names Tinogard® HS® and Cibafast® H Liquid), and polyester-8 (commercially available from HallStar Co. under the trade name Polycrylene®). In an embodiment of the present invention the additional UV-active ingredient is butyloctyl salicylate.

The term “SPF booster” means a compound or composition that when used in a formulation in conjunction with a UV screening agent, increases the SPF of the formulation without increasing the amount of UV screening agent in the formulation. An example SPF booster capable of reflecting UV light is glass microspheres. Typically, the glass microspheres used in the compositions of the invention are essentially homogeneous and essentially uniform in sphericity and have a mean particle size of between about 5 μm and 70 μm, such as from about 10 μm to 20 μm. Glass microspheres useful in the present invention include hollow microspheres of calcium aluminum borosilicate (commercially available from Presperse Inc. under the trade name LUKSIL®), sodium borosilicate particulates (commercially available from PQ Corporation under the trade name Q-CEL 570), calcium/sodium borosilicate hollow microspheres (commercially available from 3M under the trade names ES 22 and 1K), calcium/sodium borosilicate microspheres (commercially available from 3M's under the trade name Scotchlite™ K₂₀ product).

Additional UV-active ingredients are typically employed in an amount of from about 1 to about 10% by weight, such as from about 1 to about 7% by weight, and from about 2 to about 5% by weight, based on the total weight of the composition, including all ranges and subranges therebetween.

In an embodiment of the present invention, the UV protecting composition contains: (a) from about 6% to about 8% by weight of an organic UV sunscreen active chosen from octocrylene, butyl methoxydibenzoylmethane, benzophenone-3, and mixtures thereof; (b) about 1% by weight of a semi-crystalline polymer which is solid at ambient temperature and has a melting point of less than 80° C. chosen from poly(stearyl acrylate)s and poly(behenyl acrylate)s; (c) from about 10% to about 15% by weight of hollow latex particles comprising styrene/acrylate copolymer; and (d) from about 7% to about 9% by weight of butyloctyl salicylate, all weights being based on the total weight of the composition, including all ranges and subranges therebetween.

### Additional Ingredients

The compositions of the present invention may also include additional cosmetic and dermatological ingredients.

### Inorganic UV Sunscreen Actives

The composition of the invention may include an inorganic UV screening agent. Inorganic UV sunscreen actives which may be used in accordance with the present invention are metal oxide pigments. More preferentially the inorganic sunscreen actives of the invention are metal oxide particles having an average elementary particle size of less than or equal to 500 nm, more preferably between 5 nm and 500 nm and more preferably still between 10 nm and 100 nm, and preferentially between 15 and 50 nm.

They may be selected especially from titanium oxides, zinc oxides, iron oxides, zirconium oxides, cerium oxides or mixtures thereof. The metal oxide pigments may be coated or uncoated, such as coated or uncoated titanium oxide.

Coated and uncoated metal oxide pigments of this kind are described more particularly in patent application EP-A-0,518,773. Commercial pigments that may be mentioned include the products sold by the companies Kemira, Tayca, Merck and Degussa.

Inorganic UV sunscreen actives, if present, are typically employed in an amount of from about 0.1 to about 10% by weight, such as from about 0.5 to about 5% by weight, based on the total weight of the composition.

Additional Cosmetic and/or Dermatological Actives

The compositions of the present invention may also include additional cosmetic and/or dermatological active agents including moisturizers or humectants, desquamating agents, agents improving the barrier function, depigmenting agents, antioxidants, dermo-relaxing or dermo-decontracting agents, anti-glycation agents, agents stimulating the synthesis of dermal and/or epidermal macromolecules and/or preventing their degradation, agents stimulating fibroblast or keratinocyte proliferation and/or keratinocyte differentiation, agents promoting the maturation of the horny envelope, NO-synthase inhibitors, peripheral benzodiazepine receptor (PBR) antagonists, agents increasing the activity of the sebaceous gland, agents stimulating the energy metabolism of cells, tensioning agents, fat restructuring agents, slimming agents, agents promoting the cutaneous microcirculation, calmatives and/or anti-irritants, sebo-regulating or anti-seborrheic agents, astringents, cicatrizing agents, anti-inflammatory agents and anti-acne agents.

A person skilled in the art will select the active agent or agents as a function of the effect which is desired on the skin, hair, eyelashes, eyebrows or nails. In an embodiment of the invention, additional cosmetic and/or dermatological active agents, if present, are employed in the compositions in an amount of from about 0.001% to about 20% by weight, such as from about 0.5% to 5%, and from about 0.1% to 1% by weight, based on the total weight of the composition, including all ranges and subranges therebetween.

### Complementary Ingredients

To complement and/or optimize the effects imparted by the cosmetic and/or dermatological active agents mentioned above an the keratin tissue, the compositions of the invention may further include complementary ingredients that impart an immediate visual effect that will be taken up by the biological effect of the active agents mentioned above. These complementary ingredients may also, via a mechanical action (e.g., abrasive fillers), amplify the effect of the actives mentioned above. Representative examples of such complementary ingredients include matting agents, soft-focus effect fillers, fluorescers, agents for promoting the naturally pinkish coloration of the skin, abrasive fillers or exfoliants, and mixtures thereof.

### Adjuvants

The compositions of the invention may further include any adjuvant commonly used in cosmetics, and will find application in the skin and hair care, makeup, and suntan product areas. Adjuvants useful in the compositions of the present invention include fatty substances, organic solvents, ionic or nonionic, hydrophilic or lipophilic thickeners, demulcents, humectants, opacifiers, stabilizers, emollients, silicones, antifoams, fragrances, preservatives, anionic, cationic, nonionic, zwitterionic or amphoteric surfactants, actives, fillers, polymers, propellants and alkalifying or acidifying agents, or any other ingredient commonly used in cosmetology and/or dermatology.

The fatty substances may be constituted of an oil or a wax or mixtures thereof. An oil is a compound that is liquid at ambient temperature. A wax is a compound that is solid or substantially solid at ambient temperature and whose melting point is generally greater than 35° C.

Oils that may be mentioned include mineral oils (paraffin); plant oils (sweet almond oil, macadamia oil, grapeseed oil or jojoba oil); synthetic oils, for instance perhydrosqualene, fatty alcohols or fatty amides (for instance isopropyl lauroyl sarcosinate sold under the name “ELDEW SL-205” by the company Ajinomoto), fatty acids or fatty esters (for instance the C₁₂-C₁₅ alkyl benzoate sold under the trade name “FINSOLV TN” or “WITCONOL TN” by the company Witco, phenylethyl benzoate sold under the name XTEND 226 by the company ISP or Spectrasol PEB by the company CP Hall, octyl palmitate, isopropyl lanolate and triglycerides, including capric/caprylic acid triglycerides, and dicaprylyl carbonate sold under the name “CETIOL CC” by the company Cognis), oxyethylenated or oxypropylenated fatty esters and ethers; silicone oils (cyclomethicone and polydimethylsiloxanes, or PDMS) or fluoro oils, and polyalkylenes.

Waxy compounds that may be mentioned include paraffin, carnauba wax, beeswax and hydrogenated castor oil.

Among the organic solvents that may be mentioned are lower alcohols and polyols. These polyols may be chosen from glycols and glycol ethers, for instance ethylene glycol, propylene glycol, butylene glycol, dipropylene glycol or diethylene glycol.

Hydrophilic thickeners that may be mentioned include carboxyvinyl polymers such as the Carbopol products (carbomers) and the Pemulen products (acrylate/C10-C30-alkylacrylate copolymer); polyacrylamides, for instance the crosslinked copolymers sold under the names SEPIGEL 305 (CTFA name: polyacrylamide/C13-14 isoparaffin/Laureth 7) or SIMULGEL 600 (CTFA name: acrylamide/sodium acryloyldimethyltaurate copolymer/isohexadecane/polysorbate 80) by the company SEPPIC; 2-acrylamido-2-methylpropanesulfonic acid polymers and copolymers, which are optionally crosslinked and/or neutralized, for instance the poly(2-acrylamido-2-methylpropanesulfonic acid) sold by the company Hoechst under the trade name “HOSTACERIN AMPS” (CTFA name: ammonium polyacryldimethyltauramide), cellulosic derivatives such as hydroxyethylcellulose; polysaccharides and especially gums such as xanthan gum; and mixtures thereof.

Lipophilic thickeners that may be mentioned include synthetic polymers such as the poly C₁₀-C₃₀ alkyl acrylate sold under the name “DORESCO IPA 13-1” by the company Landec, or else modified clays such as hectorite and its derivatives, for instance the products sold under the Bentone names.

As will be appreciated, a person skilled in the art will take care to select the additional ingredients and/or the amounts thereof such that the advantageous properties intrinsically associated with the compositions of the present invention are not substantially, adversely affected by the addition(s).

### Composition Forms

Generally, the compositions of the invention are cosmetic compositions intended for contacting with the surface parts of the human body. Typically, a composition of the present invention is aqueous and may have any forms that are generally used for topical application, such as an oil-in-water emulsion (direct emulsion), water-in-oil emulsion (inverse emulsion), or an aqueous gel.

The compositions according to the invention may be prepared according to techniques that are well known to those skilled in the art. They may be in particular in the form of a simple or complex emulsion (O/W, W/O, O/W/O or W/O/W) such as a cream, a milk or a cream-gel; in the form of an aqueous gel; in the form of a lotion, or a stick. They may optionally be packaged as an aerosol and may be in the form of a mousse or a spray.

The compositions according to the invention are preferably in the form of an oil-in-water or water-in-oil emulsion. Emulsions generally contain at least one emulsifier chosen from amphoteric, anionic, cationic and nonionic emulsifiers, which are used alone or as a mixture. The emulsifiers are appropriately chosen according to the emulsion to be obtained (W/O or O/W). When it is an emulsion, the aqueous phase of the emulsion may comprise a nonionic vesicular dispersion prepared according to known processes (Bangham, Standish and Watkins, J. Mol. Biol. 13, 238 (1965), FR 2 315 991 and FR 2 416 008).

The compositions according to the invention find their application in a large number of treatments, especially cosmetic treatments, of keratinous tissue, such as the skin, the lips and the hair, including the scalp, especially for protecting and/or caring for the skin, the lips and/or the hair, and/or for making up the skin and/or the lips.

Another subject of the present invention consists of the use of the compositions according to the invention as defined above for the manufacture of products for cosmetically treating the skin, lips, nails, hair, eyelashes, eyebrows and/or scalp, especially care products, antisun products and makeup products.

The cosmetic compositions according to the invention may, for example, be used as care products and/or sun protection products for the face and/or body, of liquid to semi-liquid consistency, such as lotions, milks, creams of thicker or thinner consistency, gels and cream-gels. They may optionally be packaged as an aerosol and take the form of a foam or spray.

The cosmetic compositions according to the invention may be used, for example, as makeup products.

The compositions according to the invention in vaporizable fluid lotion form in accordance with the invention are applied to the skin or the hair in the form of fine particles by means of pressurization devices. The devices in accordance with the invention are well known to those skilled in the art and comprise non-aerosol pumps or atomizers, aerosol containers comprising a propellant, and also aerosol pumps using compressed air as propellant. These pumps are described in patents U.S. Pat. No. 4,077,441 and U.S. Pat. No. 4,850,517 (which form an integral part of the content of the description).

The compositions packaged in aerosol form in accordance with the invention generally contain conventional propellants, for instance hydrofluoro compounds, dichlorodifluoromethane, difluoroethane, dimethyl ether, isobutane, n-butane, propane or trichlorofluoromethane. They are preferably present in amounts ranging from 15% to 50% by weight relative to the total weight of the composition.

The following examples are intended to further illustrate the present invention. They are not intended to limit the invention in any way. Unless otherwise indicated, all parts are by weight.

### Examples

### p53

The cellular protein, p53 is of paramount importance in the protection of cells from DNA damage. p53 is upregulated in response to UV exposure and binds to DNA to either regulate repair mechanisms or initiate apoptosis. DNA repair mechanisms are initiated when the damage is minimal. With overwhelming DNA damage, p53 initiates apoptosis, which prevents the survival of damaged cells that would otherwise propagate with mutations. Healthy skin not treated with sunscreen prior to UV exposure will have a high density of p53 expressed in tissue in order to suppress tumor formation. p53 is the most commonly mutated gene found in human cancers, particularly non-melanoma skin cancers. The lower the level of p53 expression, the better the protection from UV light.

Sunburn Cells

Sunburn cells are actually photodamaged cells that are undergoing apoptosis. Cells will undergo apoptosis when the extent of DNA damage exceeds the capability of the repair mechanisms. Thus, the appearance of sunburn cells is a marker for DNA damage—the lower the number of sunburn cells, the better the protection from DNA damage.

Experimental Protocol

Subjects, “n,” where n is 5 or 10, (male and female, age 18-60 years), (Fitzpatrick skin types I, II and III) were recruited for the study. The subjects were randomized for formula application.

On Day 1, four areas (7.5 cm×5 cm) were marked on the hairless lower back skin of each subject. A dose of 75 μl of each of the formulas was applied (2 mg/cm2) daily for four (4) consecutive days on two assigned sites. Two sites were left untreated.

On Day 3, the minimal erythema dose (MED) was determined for each subject. Six separate sites near the treatment area were exposed to a range of doses (20 to 60 mJ/cm2 at 10 mJ/cm2 intervals).

On Day 4, the MED was determined as the spot receiving the lowest dose with erythema extending to the borders. On Day 4, three test sites were exposed to 5xMEDs of solar-simulated UV-radiation. One of the untreated sites was not exposed.

On Day 5, four-millimeter (4-mm) punch biopsies, were collected from the two treated sites, one Untreated/Exposed site (positive control) and an Untreated/Unexposed site (negative control) from each subject and fixed in 10% buffered formalin. Biopsy specimens were processed for immunohistochemistry of the biomarker.

Immunohistochemistry: Antibodies used were mouse monoclonal antibodies made against human antigens.

Formalin-fixed, paraffin-embedded tissues were sectioned at five μm, mounted on adhesive slides, and immunostained manually using standard methods. After de-paraffinization of the slides, endogenous peroxidase was quenched, and the slides were subjected to heat-induced epitope retrieval in 1 mM EDTA for 30 minutes using a vegetable steamer. The slides were then immersed in previously-optimized dilutions of mouse monoclonal anti-p53 antibody (clone DO7, Immunovision) and incubated for 20 min at 25° C. Following this, the slides were immersed in the appropriate peroxidase-conjugated polymer reagent (Vision BioSystems, Norwell, Mass.) for 45 minutes at 25° C. The reaction product was developed by immersing the slides in prepared diaminobenzidine solution (Invitrogen, Carlsbad, Calif.) at 25° C. for five minutes, followed by enhancement in 0.5% copper sulfate, dehydration, and counterstaining with hematoxylin. Following immunostaining, total number of p53 positive cells was quantified in epidermis and results expressed as mean # cells/mm.

Sunburn Cells: Procedure was identical as described above using mouse monoclonal anti-sunburn cell antibody. Following immunostaining, total number of sunburn cells was quantified in epidermis and results expressed as mean # cells/mm.

UV Source

The UVR was supplied by single port solar simulator (Model 16S, Solar UV Simulator, Solar Light Co., Philadelphia) including a 150 watt xenon arc lamp and UG-11/1 mm and WG-320 filters (Schott Glass Technologies) to deliver a 290-400 nm ultraviolet range comparable to that of natural solar spectrum. At a distance of approximately 6.5 cm from the lamp housing, the exposed surface was exposed to a 1.0 cm diameter spot UVR. Exposures were performed by varying the time of exposure (in seconds) while keeping the energy level constant. Opening and closing of the light shutter was performed manually. The radiation output of the xenon bulb was measured using the 3D-600 meter (Solar Light Co.). The relative UVB and UVA output of the solar simulator under the conditions of the experiment was determined to be approximately 4.1 mW/cm2, and the relative output of UVA and UVB was approximately 88.9% of UVA and 11.1% UVB.

TABLE 1
Formula Compositions (% w/w)

| | Example 1 | Example 2 |
|---|---|---|
| UV Filters | 34.0%¹ | 8.0%² |
| SunSpheres ™³ | 4.0% | 15.0% |
| Boosters | 3.2% | 8.5% |
| Carrier | QS | QS |
| SPF value (in-vitro) | 61.5⁴ | 46 |

¹Example 1 UV Filters 34%
Oxybenzone 6%
Octisalate 5%
Octocrylene 5%
Homosalate 15%
Avobenzone 3%
²Example 2 UV Filters 8%
Octocrylene 6%
Avobenzone 2%
³Sunspheres ™ are hollow latex particles made from Styrene/Acrylates copolymer, and commercially available from Rohm & Haas.
⁴The FDA approved in-vivo method SPF value was 69. That the in-vitro SPF value of 61.5 is very similar to that of the in-vivo SPF value of 69 indicates the high level of accuracy in the methodology used to determine the in-vitro SPF values described herein.

As seen from the above Table 1, Example 1 is similar to a traditional sun protection formula having 34% by weight of UV filters and minimal amounts of styrene/acrylates copolymers and SPF boosters. In contrast, the inventive Example 2 has minimal amounts of UV filters at 8% by weight, and increased amounts of styrene/acrylates copolymers and SPF boosters.

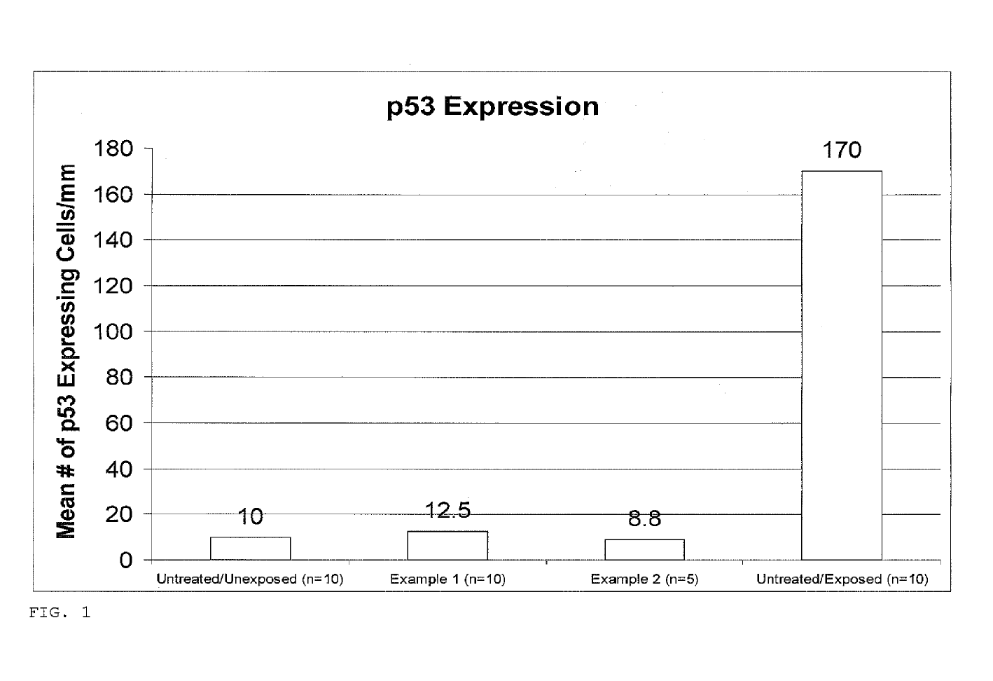

As can be seen in FIG. 1, the level of p53 expression is shown with respect to the four sites mentioned above: Untreated/Unexposed; treated with Example formulas 1 and 2; and Untreated/Exposed.

The two treated sites are at parity with the Untreated/Unexposed site, evidencing the efficacy of the two example formulas. The Untreated/Exposed site shows a considerably higher level of p53 expression than the other 3 sites. Thus, the two example formulas provide a significant level of protection against UV light. Further, when comparing the level of p53 expression between the traditional formula Example 1 and the inventive formula Example 2, it can be seen that the inventive Example 2 is as effective as the traditional Example 1. Thus, at least the same level of protection is provided using significantly lower amounts of organic UV filters.

The SPF value for Example 1 is 69, determined using the FDA approved in-vivo method. The SPF value of Example 1 is 61.5, determined in-vitro. That the two SPF values are similar indicates the level of accuracy of the in-vitro method used to determine the SPF values of both the example formulas. The SPF value of Example 2 is 46, determined in-vitro. Thus, even at a lower SPF value of 46, the inventive Example 2 is highly efficacious in providing protection from UV light as evidenced by the low level of p53 expression.

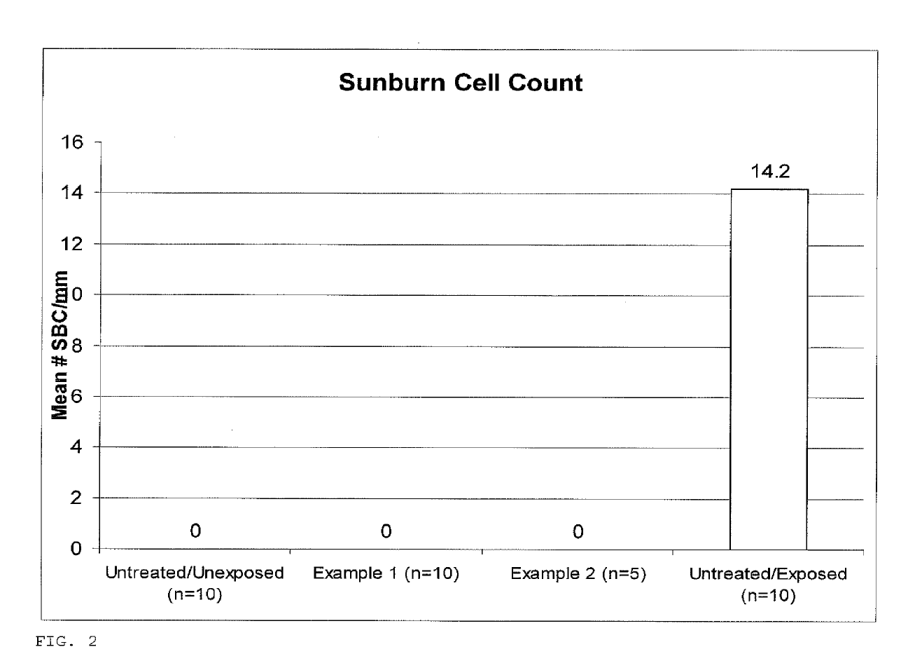

As can be seen in FIG. 2, the number of sunburn cells is shown with respect to the four sites mentioned above: Untreated/Unexposed; treated with Example formulas 1 and 2; and Untreated/Exposed. The treated sites have no sunburn cell damage and are at parity with the Untreated/Unexposed site, once again evidencing the efficacy of the example formulas. In contrast, the Untreated/Exposed site has a much higher number of sunburn cells. Further, as explained above, the inventive Example 2 is as effective as the traditional Example 1, using a lower level of traditional UV filters.

Here too, it can be seen that even at a lower SPF value, the inventive Example 2 is highly efficacious in minimizing the number of sunburn cells.

Thus, it has been surprisingly discovered that high, efficacious levels of sun protection are achieved when employing styrene/acrylates copolymers in an amount greater than or equal to the amount of organic UV filters present in the composition.

All publications cited in the specification, both patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A UV protecting composition comprising:
(a) at least one organic UV sunscreen active;
(b) at least one semi-crystalline polymer which is solid at ambient temperature and has a melting point of less than about 80° C.;
(c) hollow latex particles; and
(d) at least one additional ingredient chosen from:
i) a UV light absorbing compound having an SPF of less than 2, and
ii) an SPF booster capable of reflecting UV light, different from (c),
wherein (c) is employed in an amount equal to, or greater than, the amount of (a) present in the composition.

2. The composition of claim 1, wherein (a) is chosen from:
Ethylhexyl Methoxycinnamate,
Ethylhexyl Salicylate,
Homosalate,
Butyl Methoxydibenzoylmethane,
Octocrylene,
Phenylbenzimidazole Sulfonic Acid,
Benzophenone-3,
Benzophenone-4,
Benzophenone-5,
n-Hexyl 2-(4-diethylamino-2-hydroxybenzoyl)benzoate,
4-Methylbenzylidene camphor,
Terephthalylidene Dicamphor Sulfonic Acid,
Disodium Phenyl Dibenzimidazole Tetrasulfonate,
Methylene bis-Benzotriazolyl Tetramethylbutylphenol,
Bis-Ethylhexyloxyphenol Methoxyphenyl Triazine,
Ethylhexyl Triazone,
Diethylhexyl Butamido Triazone,
2,4,6-Tris(dineopentyl 4′-aminobenzalmalonate)-s-triazine,
2,4,6-Tris(diisobutyl 4′-aminobenzalmalonate)-s-triazine,
2,4-Bis(n-butyl 4′-aminobenzoate)-6-(aminopropyltrisiloxane)-s-triazine,
2,4-Bis(dineopentyl 4′-aminobenzalmalonate)-6-(n-butyl 4′-aminobenzoate)-s-triazine,
2,4,6-Tris(biphenyl-4-yl)-1,3,5-triazine,
2,4,6-Tris(terphenyl)-1,3,5-triazine,
Drometrizole Trisiloxane,
Polysilicone-15,
1,1-Dicarboxy(2,2′-dimethylpropyl)-4,4-diphenylbutadiene,
2,4-Bis[5-1-(dimethylpropyl)benzoxazol-2-yl(4-phenyl)imino]-6-(2-ethylhexyl)-imino-1,3,5-triazine,
and mixtures thereof.

3. The composition of claim 1, wherein (a) is chosen from octocrylene, butyl methoxydibenzoylmethane, benzophenone-3, and mixtures thereof.

4. The composition of claim 1, wherein (a) is present in an amount of from about 1% to about 15% by weight, based on the total weight of the composition.

5. The composition of claim 1, wherein (b) is chosen from saturated C₁₄ to C₂₂ alkyl (meth)acrylates.

6. The composition of claim 1, wherein (b) is chosen from poly(stearyl acrylate)s and poly(behenyl acrylate)s.

7. The composition of claim 1, wherein (b) is present in an amount of from about 0.1% to about 4% by weight, based on the total weight of the composition.

8. The composition of claim 1, wherein the particles of (c) have an average diameter of from about 100 nm to about 380 nm.

9. The composition of claim 1, wherein (c) comprises a copolymer of styrene and (meth)acrylic acid or one of its C₁-C₂₀ alkyl esters.

10. The composition of claim 1, wherein (c) comprises styrene/acrylate copolymer.

11. The composition of claim 1, wherein (c) is present in an amount of from about 1% to about 20% by weight, based on the total weight of the composition.

12. The composition of claim 1, wherein (d) is chosen from butyloctyl salicylate, diethylhexyl syringylidenemalonate, diethylhexyl 2,6-naphthalate, octadecyl di-t-butyl-4-hydroxyhydrocinnamate, sodium benzotriazolyl butylphenol sulfonate, sodium benzotriazolyl butylphenol sulfonate, polyester-8, and mixtures thereof.

13. The composition of claim 1, wherein (d) is butyloctyl salicylate.

14. The UV protecting composition of claim 1, wherein (d) is chosen from glass microspheres.

15. The composition of claim 1, wherein (d) is present in an amount of from about 1% to about 10% by weight, based on the total weight of the composition.

16. A UV protecting composition comprising:
(a) from about 6% to about 8% by weight of an organic UV sunscreen active chosen from octocrylene, butyl methoxydibenzoylmethane, benzophenone-3, and mixtures thereof;
(b) about 1% by weight of a semi-crystalline polymer which is solid at ambient temperature and has a melting point of less than 80° C. chosen from poly(stearyl acrylate)s and poly(behenyl acrylate)s;
(c) from about 10% to about 15% by weight of hollow latex particles comprising styrene/acrylate copolymer; and
(d) from about 7% to about 9% by weight of butyloctyl salicylate,
all weights being based on the total weight of the composition.

17. A process for protecting a keratinous tissue from free-radical induced damage comprising applying onto the keratinous tissue a composition comprising:
(a) at least one organic UV sunscreen active;
(b) at least one semi-crystalline polymer which is solid at ambient temperature and has a melting point of less than about 80° C.;
(c) hollow latex particles; and
(d) at least one additional ingredient chosen from:
i) a UV light absorbing compound having an SPE of less than 2, and
ii) an SPF booster capable of reflecting UV light, different from (c),
wherein (c) is employed in an amount equal to, or greater than, the amount of (a) present in the composition.
